(19) United States
(12) Patent Application Publication
(10) Pub. No.: US 2023/0310478 A1
(43) Pub. Date: Oct. 5, 2023

(54) METHODS AND COMPOSITIONS FOR THE TREATMENT OF PULMONARY HYPERTENSION AND CANCER

(71) Applicant: Ann and Robert H. Lurie Children's Hospital of Chicago, Chicago, IL (US)

(72) Inventor: Youyang Zhao, Deerfield, IL (US)

(21) Appl. No.: 18/043,831

(22) PCT Filed: Sep. 2, 2021
(86) PCT No.: PCT/US21/48868

§ 371 Date: Mar. 2, 2023

Related U.S. Application Data
(60) Provisional application No. 63/073,770, filed on Sep. 2, 2020.

(51) Int. Cl. A61K 31/7048; A61K 31/4748; A61K 31/473; A61K 31/352; A61K 31/4745; A61K 45/06; A61P 9/12

(52) U.S. Cl. CPC A61K 31/7048; A61K 31/4748; A61K 31/473; A61K 31/352; A61K 31/4745; A61K 45/06; A61P 9/12

(57) ABSTRACT

Disclosed herein are compositions and methods useful for the treatment of pulmonary hypertension, such as pulmonary arterial hypertension (PAH), in a subject in need thereof. Also disclosed herein are compositions and methods useful for the treatment of cancers characterized by an increase in HIT-2 a expression and/or activity in a subject in need thereof. In some embodiments, the methods include administering a therapeutic composition comprising one or more of ethacridine, propantheline, etoposide, irinotecan, quinidine, and hydroxy ethylapoquimne (HEAQ), and/or an analog thereof.

### CROSS-REFERENCE TO RELATED APPLICATIONS

This application claims the benefit of U.S. Provisional Application No. 63/073,770 filed on Sep. 2, 2020, which is incorporated herein by reference in its entirety.

### STATEMENT REGARDING FEDERALLY SPONSORED RESEARCH OR DEVELOPMENT

This invention was made with government support under HL133951 and HL140409 awarded by the National Institutes of Health. The government has certain rights in the invention.

### BACKGROUND

Pulmonary hypertension (PH), including pulmonary artery hypertension (PAH), is characterized by excessive vasoconstriction and obliterative vascular remodeling that leads to right heart failure (RHF) and death. The histopathological features of PAH include thickening of the medial and adventitial layers of pulmonary vasculature, and the formation of vascular occlusions and plexiform lesions. Although significant advances have been made in understanding the pathogenesis of PAH in the past decade, no effective therapy has been developed to reverse pulmonary vascular remodeling, inhibit RHF and promote survival of PAH patients. Current therapies mainly target the abnormalities in vasoconstriction in the prostacyclin, nitric oxide and endothelin signaling pathways, but not the obliterative vascular remodeling abnormalities. Hence, only modest improvements are achieved in PAH morbidity and mortality. Therefore, novel therapeutic agents are urgently needed for PAH patients. Recently studies have demonstrated the important role of HIF-2α activation in the pathogenesis of obliterative vascular remodeling and PAH. Targeting HIF-2α may represent a novel and effective therapy of PAH in patients.

HIF-1α and HIF-2α have been recognized as a key protein regulating the transcription of multiple genes related to erythropoiesis, angiogenesis, glycolysis, vasodilation, and cancer. After exposure of normal and cancer cells to hypoxia, a rapid increase of HIF-1α and HIF-2α heterodimerization with the HIF1b protein (ARNT) occurs, leading to increasing amounts of the HIF1 and HIF2 protein in the nucleus. Although stabilization of HIF proteins may occur because of mutations or oncogene activation, HIF overexpression in tumors is likely to reflect pronounced tumor hypoxia. Tumor hypoxia is a well-recognized factor linked to poor response to radiotherapy, but it is also linked to a poor response to chemotherapy, and has been known to hinder the efficacy of photodynamic therapy as a cancer treatment. Therefore, novel therapeutic agents are urgently needed for patients suffering from HIF-2α overexpressing cancers.

### SUMMARY

Disclosed herein are compositions and methods useful for the treatment of pulmonary hypertension, such as pulmonary arterial hypertension (PAH) in a subject in need thereof. Also disclosed herein are compositions and methods useful for the treatment of cancers characterized by an increase in HIF-2α expression in a subject in need thereof. In some embodiments, the methods include administering a therapeutic composition comprising one or more of ethacridine, quinidine or an analog of quinidine such as hydroxyethylapoquinine (HEAQ), etoposide, methantheline/propantheline, and irinotecan or analogs thereof.

### BRIEF DESCRIPTION OF THE DRAWINGS

FIG. 1A-B. Quinidine is a potent HIF-2α inhibitor. (A) Luciferase assay showing that Quinidine repressed HRE luciferase (Luc) activity in a dose-dependent manner in 786-O cells. HRE, hypoxia-responsive element. (B) Quinidine didn't repress control SV40 luciferase activity in 786-O cells indicating no cell toxic effects at the doses tested. ***P<0.001; one-way ANOVA with Tukey post hoc analysis for multiple group comparisons.

FIG. 2A-C. On target of HIF-2α by Quinidine in human lung endothelial cells. (A) Luciferase activity assay showing Quinidine suppression of HRE activity in DMOG-treated human lung microvascular endothelial cells (HLMVECs). HLMVECs were treated with DMOG (1 mM) to activate HIF signaling. Right after DMOG treatment, Quinidine (Q, 20 μM) or Vehicle (Veh, DMSO) was added to DMOG-treated cells for 24 hours. (B) Quantitative RT-PCR analysis demonstrating inhibited expression of HIF-2α target genes in PHD2-deficient HLMVECs by Quinidine treatment. HLMVECs were transfected with PHD2 siRNA (siPHD2) or control scrambled RNA (siCtl). At 48 hours post-transfection, the cells were treated with Quinidine (Q, 20 μM) or vehicle (DMSO) for 24 hours. (C) Quinidine did not affect expression of HIF-1α target genes in DMOG-treated human PASMCs, indicating Quinidine is a HIF-2α-selective inhibitor. Q=Quinidine. n.s., not significant. **P<0.01; and ***P<0.001; one-way ANOVA with Tukey post hoc analysis (A-C).

FIG. 3A-F. Quinidine treatment inhibited PAH in MCT-challenged rats. (A) A diagram showing the experimental timeline of Quinidine treatment in MCT-challenged rats. Two weeks post-MCT challenge, rats were treated with either Quinidine or vehicle for 14 days. (B, C) Quinidine treatment (s.c. osmotic pump, 10-12 mg/kg/day) reduced RVSP (B) and RV hypertrophy (C) in MCT rats. (D) Restoration of pulmonary artery (PA) AT/ET ratio indicative of normalized pulmonary arterial function by Quinidine treatment. (E) Echocardiography-derived measurements demonstrating reduction in RV wall thickness by Quinidine treatment in MCT rats. (F) Improved RV contractility by Quinidine treatment. **P<0.01, and ***P<0.001; one-way ANOVA with Tukey post hoc analysis for multiple group comparisons (B-F).

FIG. 4A-F. Quinidine treatment reversed pulmonary vascular remodeling in MCT-treated rats and promoted survival. (A) Representative micrographs of Russel-Movat pentachrome staining of MCT-rat lungs. Arrows point to occlusive vessels (thickened wall). (B) Quantification of PA wall thickness in MCT rat lungs. (C, D) Marked decrease in muscularization of distal pulmonary arterioles (<75 μm in diameter) in Quinidine-treated MCT rats compared to vehicle-treated rats. Lung sections were immunostained with anti-α-smooth muscle actin (α-SMA) antibody (red); nuclei were counterstained with DAPI (blue). Arrows indicate muscularized small vessels (<75 μm). (E) Quantitative RT-PCR analysis of HIF-2α downstream target genes showing Quinidine suppressed HIF-2α activation in MCT-rat lungs. (F) Quinidine treatment promoted survival. *P<0.05, **P<0.01, and ***P<0.001; one-way ANOVA with Tukey post hoc analysis for multiple group comparisons (B, D and E); and Log-rank (Mantel-Cox) test (F). Scale bars: 50 μm.

FIG. 5. Inhibitory activity of Quinidine analogs in 786-O cells. At 24 h post-treatment with Quinidine (Q) and its analogs, hydroxyethylquinine (HEQ), HEAQ, hydroxyethylquinidine (HEQD), luciferase activity was determined. PBS was used in CTL and compound C76 was used as a positive control. **, P<0.01, ****, P<0.0001. Student t test.

FIG. 6. HEAQ treatment markedly reduced RVSP in MCT rats. At 14 days post-MCT challenge, the rats were treated with HEAQ (20 mg/kg, oral, daily) or PBS for another 14 days. RVSP was then measured. *P<0.05 (n=3), Student t test.

FIG. 7. Ethacridine is a potent HIF-α inhibitor. To validate the inhibitory effect of Ethacridine, stable 786-O cells expressing HRE-Luciferase was treated with Ethacridine at the indicated concentration for 24 h. Luciferase activity was then measured and normalized to control vehicle (DMSO) group. Ethacridine at 5 uM could inhibited approximately 80% HRE activity, i.e., HIF activity. Data are expressed as mean±SD.

FIG. 8A-B. Combination treatment with low doses of Quinidine and Ethacridine effectively inhibited PH in MCT rats. At 14 days post-MCT challenge, the rats were treated with Quinidine (10 mg/kg, oral, twice a day) and Ethacridine (7.5 mg/kg, i.p., twice a day) (Q+E) at the same time for 14 days. Separate groups of rats were treated with either Quinidine (10 mg/kg, oral, twice a day) or Ethacridine (7.5 and 1.5 mg/kg, i.p., twice a day). RVSP was measured (A) and RV hypertrophy was calculated (B) at 28 days post-treatment. Vehicle=PBS. **P<0.01. One-way ANOVA.

FIG. 9A-B. Combination treatment with low doses of Propantheline and Ethacridine effectively inhibited PH in MCT rats. At 14 days post-MCT challenge, the rats were treated with Propantheline (1.0 mg/kg, i.p., twice a day) and Ethacridine (7.5 mg/kg, i.p., twice a day) (P+E) at the same time for 14 days. A separate group of rats was treated with either Propantheline (2.5 mg/kg, i.p., twice a day). RVSP was measured (A) and RV hypertrophy was calculated (B) at 28 days post-treatment. Vehicle=PB S. **P<0.01, ****P<0.0001. One-way ANOVA.

### DETAILED DESCRIPTION

The present invention is described herein using several definitions, as set forth below and throughout the application.

### Definitions

Unless otherwise specified or indicated by context, the terms “a”, “an”, and “the” mean “one or more.” For example, “an inhibitor of PAH” should be interpreted to mean “one or more inhibitors of PAH.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the term “subject” may be used interchangeably with the term “patient” or “individual” and may include an “animal” and in particular a “mammal.” Mammalian subjects may include humans and other primates, domestic animals, farm animals, and companion animals such as dogs, cats, guinea pigs, rabbits, rats, mice, horses, cattle, cows, and the like.

In some embodiments, a subject may be in need of treatment, for example, treatment may include administering a therapeutic amount of one or more agents that inhibits, alleviates, or reduces the signs or symptoms of PAH.

As used herein, the phrase “effective amount” shall mean that drug dosage that provides the specific pharmacological response for which the drug is administered in a significant number of patients in need of such treatment. An effective amount of a drug that is administered to a particular patient in a particular instance will not always be effective in treating the conditions/diseases described herein, even though such dosage is deemed to be a therapeutically effective amount by those of skill in the art.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

Pharmaceutical or therapeutic compositions disclosed herein may be formulated for administration by any suitable route of delivery, such as oral, parenteral, rectal, nasal, topical, or ocular routes, or by inhalation. By way of example but not by way of limitation, PAH inhibitory therapeutic compositions may be administered intranasally or by inhalation.

A “subject in need of treatment” may include a subject diagnosed with, suspected of having, or at risk of pulmonary hypertension (PH), such as pulmonary arterial hypertension (PAH). In some embodiments, a subject in need thereof, may include a subject exhibiting one or more of the following symptoms: shortness of breath (dyspnea), chest pain, fatigue, dizziness or fainting spells (syncope), swelling in the ankles, legs and eventually the abdomen, cyanosis (bluish color to lips and skin), racing pulse or heart palpitations, and/or histopathological features including but not limited to thickening of the medial and adventitial layers of pulmonary vasculature, and the formation of vascular occlusions and plexiform lesions. In some embodiments, a subject in need thereof exhibits one or more of the following symptoms: increased right ventricular systolic pressure (RVSP) and mean pulmonary arterial pressure (mPAP), right ventricular hypertrophy and dilated cardiopathy and heart failure; reduced pulmonary artery function (decreased AT/ET ratio). In some embodiments, a subject in need thereof may include a subject diagnosed with, suspected of having, or at risk of one or more of genetic mutation (heritable pulmonary arterial hypertension) including HIF2α activation mutation, EGLN1 inactivation mutation, VHL dysfunction mutation, congestive heart failure, blood clots in the lungs, infection (viral, e.g., HIV, bacterial, or fungal), drug use (e.g., methamphetamines, cocaine, diet drugs, selective serotonin reuptake inhibitors (SSRIs) often used to treat depression and anxiety, and other drugs), liver disease, autoimmune diseases such as but not limited to lupus, scleroderma, and rheumatoid arthritis, congenital heart defect, lung disease such as, but not limited to emphysema, chronic bronchitis, COPD, or pulmonary fibrosis, sleep apnea, long term exposure to high altitude, chronic blood clots or clotting disorders, blood disorders including polycythemia vera and essential thrombocythemia, inflammatory disorders such as sarcoidosis and vasculitis, metabolic disorders including glycogen storage disease, kidney disease, tumors pressing against pulmonary arteries, and Eisenmenger syndrome. A disease caused by HIF-2α overexpression and/or overactivation or PHD2 deficiency and/or inactivation, or VHL (Von Hippel-Lindau tumor suppressor protein) dysfunction.

A “subject in need of treatment” may include a subject having cancer, in particular an HIF-2α overexpressing and/or overactivation cancer, an PHD (including PHD1, 2, 3) deficient or inactivation cancer, or a VHL dysfunction caner. Thus in some embodiments, a subject in need thereof may include a subject diagnosed with one or more of astrocytoma, breast, cervical, colorectal, glioblastoma including glioblastoma multiforma (GBM), head/neck cancer, hepatocellular, lung, melanoma, neuroblastoma, ovarian, prostate, clear cell renal cell carcinoma, pheochromocytoma, and paraganglioma.

As used herein, the term “reference level” refers to the level of expression of a biomarker in a known sample against which another test sample is compared. A reference level can be obtained, for example, from a known cancer sample from a different individual or group of individuals (e.g., not the individual being tested). The reference level may be determined before and/or after a treatment, e.g., from samples obtained from the same subject before and/or after treatment. A known sample can also be obtained by pooling samples from a plurality of individuals to produce a reference level over an averaged population. A “level” can be an amount of nucleic acid expression, protein expression, level of a biomarker, or a percentage of cells expressing a biomarker, e.g., in a stained, biopsy sample.

As used herein “control,” as in “control subject” or “control sample” has its ordinary meaning in the art, and refers to a sample, or a subject, that is appropriately matched to the test subject or test sample and is treated or not treated as appropriate.

Pulmonary arterial hypertension (PAH) is characterized by occlusive vascular remodeling in the lungs and progressive increases of pulmonary vascular resistance that cause right heart failure and premature death. The histopathological features of severe PAH include intima and media thickness, muscularization of distal pulmonary arteries, vascular occlusion and complex plexiform lesions. Current therapies of vasodilators have not targeted the fundamental disease modifying mechanisms and have only resulted in a modest improvement in the morbidity and mortality and the ultimate treatment remains lung transplantation. Our recent study has identified the first mouse model of PAH with Tie2Cre-mediated deletion of Egln1 (encoding hypoxia-inducible factor (HIF) prolyl hydroxylase 2, PHD2) which recapitulates many pathological features of clinical PAH including occlusive vascular remodeling and right heart failure. We also show that Hypoxia inducible factor-2a (HIF-2a) but not HIF-1a activation secondary to PHD2 deficiency mediates the severe PAH. Pharmacological inhibition of HIF-2a inhibits pulmonary vascular remodeling and right heart failure in animal models of PAH. Our screening of the FDA-approved drug library has identified several drugs as potent HIF-2a-inhibitors.

Herein, we show that treatment either quinidine or HEAQ reversed PAH in monocrotaline (MCT) rats, a well-known animal model of PAH. Furthermore, quinidine and ethacridine combination therapy synergistically inhibited PH. Also combination therapy of ethacridine and propantheline synergistically inhibited PH. Thus, quinidine or its analog alone, ethacridine or its analog alone, propantheline or its analog alone, etoposide or its analog alone, irinotecan or its analog alone as well as their combination such as quinidine and ethacridine, quinidine and etoposide, quinidine and irinotecan, ethacridine and propantheline, ethacridine and etoposide, ethacridine and irinotecan, etoposide and irinotecan, etoposide and propantheline, irinotecan and propantheline in combination or combination of their analogs can be repurposed for treatment of PAH, and potentially other forms of pulmonary hypertension, in patients in need thereof. Given that aberrant activation of HIF-2α is also a crucial driver of renal cell carcinoma (RCC) and glioblastoma multiforme (GBM), these drugs or their analogs or drug combination can be repurposed for treatment of cancer including ccRCC and GBM as well as pheochromocytomas and paragangliomas and other forms of cancer, in patients in need thereof.

### Pulmonary Hypertension

Pulmonary hypertension is a type of high blood pressure that affects the arteries in the lungs and the right side of your heart. In one form of pulmonary hypertension, called pulmonary arterial hypertension (PAH, type I), blood vessels in the lungs are narrowed, blocked or destroyed. The damage slows blood flow through the lungs, and blood pressure in the lung arteries rises. Your heart must work harder to pump blood through the lungs. The extra effort eventually causes the heart muscle to become weak and fail.

In some people, pulmonary hypertension slowly gets worse and can be life-threatening. Although there is no cure for pulmonary arterial hypertension, treatment can help reduce symptoms and improve your quality of life. But, the 5-year mortality rate is still 40-60%.

Histopathological features of PAH include, but are not limited to one or more of thickening of the medial and adventitial layers of pulmonary vasculature, and the formation of vascular occlusions and plexiform lesions.

Typical symptoms of PAH include but are not limited to shortness of breath (dyspnea), chest pain, fatigue, dizziness or fainting spells (syncope), swelling in the ankles, legs and eventually the abdomen, cyanosis (bluish color to lips and skin), racing pulse or heart palpitations, increased right ventricular systolic pressure (RVSP), right ventricular hypertrophy; reduced pulmonary artery AT/ET ratio; increased right ventricular wall thickness; reduced right ventricular contractility.

Causes include but are not limited to one or more of idiopathic (unknown cause), genetic mutation (heritable pulmonary arterial hypertension), congestive heart failure, blood clots in the lungs, infection (viral, e.g., HIV, bacterial, or fungal), drug use (e.g., methamphetamines, cocaine, diet drugs, selective serotonin reuptake inhibitors (SSRIs) often used to treat depression and anxiety, and other drugs), liver disease, autoimmune diseases such as but not limited to lupus, scleroderma, and rheumatoid arthritis, congenital heart defect, lung disease such as, but not limited to emphysema, chronic bronchitis, COPD, or pulmonary fibrosis, sleep apnea, long term exposure to high altitude, chronic blood clots or clotting disorders, blood disorders including polycythemia vera and essential thrombocythemia, inflammatory disorders such as sarcoidosis and vasculitis, metabolic disorders including glycogen storage disease, kidney disease, tumors pressing against pulmonary arteries, and Eisenmenger syndrome.

Testing for and diagnosing pulmonary hypertension, including PAH, includes but is not limited to one identifying one or more clinical symptoms (e.g., chest pain, fatigue, passing out, swelling in the ankles and legs, etc.), echocardiography, CT scan, ventilation-perfusion scan (V/Q scan), electrocardiogram (EKG or ECG), chest X-ray, exercise testing, blood work (e.g., to test for infection, autoimmune disease, etc.), and heart catheterization. Likewise, monitoring a subject during or after a course of therapy/treatment can be performed by 6-minute walking distance and using one or more of the above methods (e.g., monitoring clinical symptoms, evaluating testing and scanning results, etc.).

Current treatment often depends on the cause of pulmonary hypertension, such as PAH, in each patient and can include one or more of oxygen therapy, blood thinners, calcium channel blockers, and vasodilators, ambrisentan (Letairis), bocentan (Tracleer), macitentan (Opsumit), riociguat (Adempas), selexipag (Uptravi), sildenafil (Revatio), tadalafil (Adcirca), treprostinil (Orenitram), iloprost tromethamine (Ventavis), treprostinil (Tyvaso), epopostenol sodium (Flolan, Veletri), treprostinil. In some cases, lung transplant or atrial septostomy is recommended.

In some instances current therapies mainly target the abnormalities in vasoconstriction in the prostacyclin, nitric oxide and endothelin signaling pathways, but not the obliterative vascular remodeling abnormalities.

### Cancer

Some cancers are characterized by an increased expression or activity of HIF-2α or by a decreased expression or activity of PHDs, or VHL dysfunction and the compositions and methods disclosed herein may be useful to treat such cancers. By way of example, but not by way limitation, such cancers include clear cell renal cell carcinoma, glioblastoma including glioblastoma multiforme (GBM), astrocytoma, breast, cervical, colorectal, head/neck cancer, liver cancer, lung cancer, melanoma, neuroblastoma, ovarian cancer, prostate cancer, pheochromocytoma, and paraganglioma.

Cancer treatments and therapies include, but are not limited to radiation, chemotherapy, immunotherapy, hormone therapy, stem cell transplant, and surgery. The efficacy of a cancer treatment regimen may be monitored by evaluating clinical symptoms, and scanning for cancer/tumor size, appearance, and spread.

### Therapeutic Compositions, Formulations, and Modes of Administration

Disclosed herein are compositions and methods useful for the treatment of pulmonary hypertension, such as pulmonary arterial hypertension (PAH) in a subject in need thereof. Also disclosed herein are compositions and methods useful for the treatment of cancers characterized by an increase in HIF-2α expression and/or activity in a subject in need thereof. In some embodiments, a subject in need thereof is diagnosed with, at risk for, or suspected of having PH or cancer with HIF-2α over-expressing and/or overactivation, PHD deficiency and/or inactivation, or VHL dysfunction. In some embodiments, the compositions and methods are useful in treating a subject exhibiting one or more of the following symptoms: shortness of breath (dyspnea), chest pain, fatigue, dizziness or fainting spells (syncope), swelling in the ankles, legs and eventually the abdomen, cyanosis (bluish color to lips and skin), racing pulse or heart palpitations, increased right ventricular systolic pressure (RVSP), right ventricular hypertrophy; reduced pulmonary artery AT/ET ratio (function); increased right ventricular wall thickness and contractility.

The methods disclosed herein include administering one or more therapeutic compositions to a subject in need thereof.

In some embodiments, the compositions of the present disclosure (e.g., pharmaceutical compositions) include one or more compounds that inhibit HIF-2α activity. By way of example, but not by way of limitation, such compounds include quinidine and analogs thereof, and ethacridine and analogs thereof, propantheline/methantheline and analogs thereof, etoposide and analogs thereof, irinotecan and analog thereof. Additional HIF-2α inhibitors include, without limitation, MK-6482, PT2977, PT2385.

In some embodiments, a pharmaceutical composition of the present disclosure includes one or more HIF-2α inhibitors, and at least one additional active agent. Exemplary additional active agents include, but are not limited to blood thinners, calcium channel blockers, ambrisentan (Letairis), bocentan (Tracleer), macitentan (Opsumit), riociguat (Adempas), selexipag (Uptravi), sildenafil (Revatio), tadalafil (Adcirca), treprostinil (Orenitram), iloprost tromethamine (Ventavis), treprostinil (Tyvaso), epopostenol sodium (Flolan, Veletri), treprostinil, and cancer therapies.

As used herein the term “analogue” or “functional analogue” refer to compounds having similar physical, chemical, biochemical, or pharmacological properties. Functional analogues are not necessarily structural analogues with a similar chemical structure. An example of pharmacological functional analogues are morphine, heroine, and fentanyl, which have the same mechanism of action, but fentanyl is structurally quite different from the other two.

### Quinidine and Analogs

Quinidine with chemical formula C₂₀H₂₄N₂O₂, (2-Ethenyl-4-azabicyclo[2.2.2]oct-5-yl)-(6-methoxyquinolin-4-yl)-methanol), trade named Quinaglute, and Quinidex has the structure shown below.

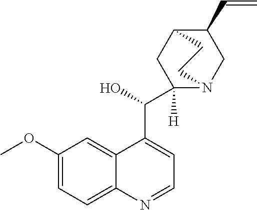 [SMILES: C=C[C@H]1C[N@]2CC[C@H]1C[C@@H]2[C@@H](O)c1ccnc2ccc(OC)cc12]

Quinidine is a medication that acts as a class I antiarrhythmic agent in the heart. It is a stereoisomer of quinine, originally derived from the bark of the cinchona tree. The drug causes increased action potential duration, as well as a prolonged QT interval. This alkaloid dampens the excitability of cardiac and skeletal muscles by blocking sodium and potassium currents across cellular membranes. It prolongs cellular action potential, and decreases automaticity. Quinidine also blocks muscarinic and alpha-adrenergic neurotransmission. Analogs of quinidine include, but are not limited to hydroxyethylapoquinine (HEAQ), hydroxyethylquinine (HEQ), and hydroxyethylquinidine (HEQD).

### Ethacridine and Analogs

Ethacridine with chemical formula C₁₅H₁₅N₃O (7-ethoxyacridine-3,9-diamine) is an aromatic organic compound based on acridine. The chemical structure of ethacridine is shown below.

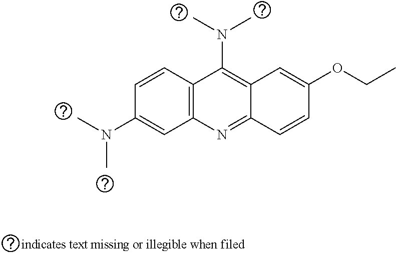 [SMILES: CCOc1ccc2nc3cc(N(C)C)ccc3c(N(C)C)c2c1]

Its primary use is as an antiseptic in solutions of 0.1%. It is effective against mostly. Gram-positive bacteria, such as Streptococci and Stpahylococci, but ineffective against Gram-negative bacteria such as Pseudomonas aeruginosa. Ethacridine is also used as an agent for second trimester abortion.

### Propantheline and Analogs

Propantheline with chemical formula C₂₃H₃₀NO₃ (methyl-di(propan-2-yl-[2-(9H-xanthene-9-carbonyloxy)ethyl]azanium). The chemical structure is shown below.

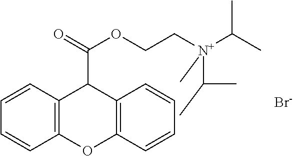 [SMILES: CC(C)[N+](C)(CCOC(=O)C1c2ccccc2Oc2ccccc21)C(C)C ; [Br-]]

Propantheline bromide (brand name Probanthine among others) is one of a group of antimuscarinic agents (including methantheline bromide) for the treatment of excessive sweating, cramps or spasms of the stomach, intestines (gut) or bladder, and involuntary urination. It can also be used to control the symptoms of irritable bowel syndrome and similar conditions.

### Etoposide and Analogs

Etoposide with chemical formula C₂₉H₃₂O₁₃ ((5S,5aR,8aR,9R)-5-[[(2R,4aR,6R,7R,8R,8aS)-7,8-dihydroxy-2-methyl-4,4a,6,7,8,8a-hexahydropyrano[3,2-d][1,3]dioxin-6-yl]oxy]-9-(4-hydroxy-3,5-dimethoxyphenyl)-5a,6,8a,9-tetrahydro-5H-[2]benzofuro[6,5-f][1,3]benzodioxol-8-one) is a beta-D-glucoside, a furonaphthodioxole and an organic heterotetracyclic compound. The chemical structure is shown below.

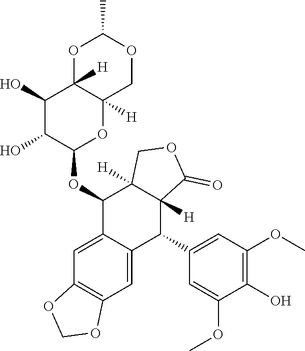

Etoposide (brand name VePesid among others) is a semisynthetic derivative of podophyllotoxin. Possessing potent antineoplastic properties, etoposide binds to and inhibits topoisomerase II and its function in ligating cleaved DNA molecules, resulting in the accumulation of single- or double-strand DNA breaks, the inhibition of DNA replication and transcription, and apoptotic cell death. Etoposide acts primarily in the G2 and S phases of the cell cycle. (NCI04)

Etoposide and teniposide are semisynthetic analogues of podophyllotoxin that are used as antineoplastic agents in the therapy of several forms of solid tumors, leukemia and lymphoma, usually in combination with other agents.

### Irinotecan and Analogs

Irinotecan with a chemical formula C₃₃H38N4O₆ ([(19S)-10,19-diethyl-19-hydroxy-14,18-dioxo-17-oxa-3,13-diazapentacyclo[11.8.0.0^{2,11.}0^{4,9}.0^{15,20}]henicosa-1(21),2,4(9),5,7,10,15(20)-heptaen-7-yl] 4-piperidin-1-ylpiperidine-1 -carboxylate; hydrochloride) is made from the natural compound camptothecin. The chemical structure is shown below.

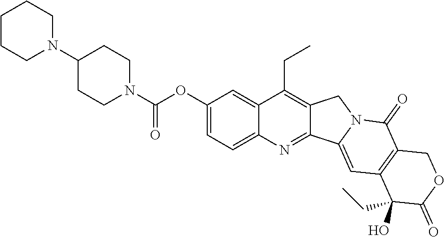 [SMILES: CCc1c2c(nc3ccc(OC(=O)N4CCC(N5CCCCC5)CC4)cc13)-c1cc3c(c(=O)n1C2)COC(=O)[C@]3(O)CC]

Irinotecan (trade name Camptosar among others) is a cytotoxic alkaloid which consists of a pentacyclic ring structure containing a pyrrole (3, 4β) quinoline moiety, an S-configured lactone form, and a carboxylate form. Irinotecan is activated by hydrolysis to SN-38, an inhibitor of topoisomerase I, leading to inhibition of both DNA replication and transcription. Irinotecan is used to treat colon cancer and small cell lung cancer.

Also disclosed herein are therapeutic compositions comprising one or a combination of HIF-2α inhibitors, and/or a combination of one or more HIF-2α inhibitors and a second active agent, or a second therapy. In some embodiments, the second active agent or therapy is useful for the treatment of PH, such as PAH. In some embodiments, the second active agent or therapy is useful for the treatment of a HIF-2α over-expressing and /or overactivation cancer.

The term “combination therapy” is used in its broadest sense and means that a subject is administered at least two agents. More particularly, the term “in combination” with respect to therapy administration refers to the concomitant administration of two (or more) active agents for the treatment of a disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms.

Further, the presently disclosed compositions can be administered alone or in combination with adjuvants that enhance stability of the agents, facilitate administration of pharmaceutical compositions containing them in certain embodiments, provide increased dissolution or dispersion, increase activity, provide adjuvant therapy, and the like, including other active ingredients. In some embodiments, such combination therapies utilize lower dosages of the conventional therapeutics, thus avoiding possible toxicity and adverse side effects incurred when those agents are used as monotherapies.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic.

The agents, whether administered alone or in combination, may be administered multiple times, and if administered as a combination, may be administered simultaneously or not, and on the same schedule or not. By way of example, a therapeutic composition may be administered multiple times per day, once per day, multiple times per week, once per week, multiple times per month, once per month, or as often as a doctor prescribes.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of an agent and at least one additional therapeutic agent is greater than the sum of the biological activities of the respective agents when administered individually. By way of example, but not by way of limitation, in some embodiments, a therapeutic composition comprises quinidine or an analog thereof such as HEAQ or HEAQ analog, or ethacridine or analog thereof alone, propantheline or its analog alone, etoposide or its analog alone, irinotecan or its analog alone or quinidine or its analog in combination with ethacridine or its analog, quinidine or its analog in combination with etoposide or its analog, quinidine or its analog in combination with irinotecan or its analog; ethacridine or its analog in combination with propantheline or its analog, ethacridine or its analog in combination with etoposide or its analog, ethacridine or its analog in combination with irinotecan or its analog; etoposide or its analog in combination with propantheline or its analog, etoposide or its analog in combination with irinotescan or its analog; propantheline or its analog in combination with irinotecan or its analog. In some embodiments, such compositions are useful for treating PH, such as PAH, or cancers, such as HIF-2α over-expressing cancers including ccRCC, GBM and others.

In therapeutic applications, the compounds of the disclosure can be formulated for a variety of modes of administration, including systemic and topical or localized administration. Such modes of administration include but are not limited to formulations for oral, parenteral, iv, inhalation, intranasal, and direct injection or administration to a tumor. Techniques and formulations generally may be found in Remington: The Science and Practice of Pharmacy (20^{th} ed.) Lippincott, Williams and Wilkins (2000).

Use of pharmaceutically acceptable inert carriers to formulate the compounds herein disclosed for the practice of the disclosure into dosages suitable for administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, dragees, liquids, gels, syrups, slurries, suspensions, and the like, for oral ingestion. For nasal or inhalation delivery, the compositions of the disclosure also may be formulated by methods known to those of skill in the art, and may include, for example, but not limited to, sprays, inhalers, vapors; solubilizing, diluting, or dispersing substances, such as saline, preservatives, such as benzyl alcohol; absorption promoters; and fluorocarbons may be included.

Pharmaceutical compositions suitable for use in the present disclosure include compositions wherein the active ingredients are contained in an effective amount to achieve its intended purpose. By way of example but not by way of limitation, in some embodiments, a pharmaceutical composition of the present disclosure comprises one or more of ethacridine, propantheline, etoposide, irinotecan, quinidine, and an analog of quinidine, such as HEAQ or HEAQ analog. In some embodiments, a pharmaceutical composition comprises one or more additional active agents, or inter carriers for formulation.

Dosage and administration of HIF-2α inhibitors, alone or in combination with additional therapeutic agents can be determined by a skilled artisan using methods that are standard in the art, based on patient age, weight, sex, race, overall health, stage of the disease. Determination of the effective amounts is well within the capability of those skilled in the art, especially in light of the detailed disclosure provided herein.

Generally, the compounds according to the disclosure are effective over a wide dosage range. For example, in the treatment of adult humans, dosages from 0.1 to 1000 mg/kg, from 0.5 to 200 mg/kg, from 1 to 50 mg per day/kg, and from 5 to 40 mg/kg per day are examples of dosages that may be used. A non-limiting dosage is 5 to 30 mg/kg per day. In some exemplary embodiments, the dosage for ethacridine alone is 0.1-100 mg/kg, a non-limiting dosage is 1-10 mg/kg per day. The dosage for quinidine is 0.1-100 mg/kg, a non-limiting dosage is 1-10 mg/kg per day. In some exemplary embodiments, the dosage for HEAQ is 0.1-100 mg/kg, a non-limiting dosage is 1-10 mg/kg per day. In some exemplary embodiments, the dosage for propantheline alone is 0.1-100 mg/kg, a non-limiting dosage is 0.1-10 mg/kg per day. In some exemplary embodiments, the dosage for etoposide alone is 0.1-100 mg/kg, a non-limiting dosage is 0.5-10 mg/kg per day. In some exemplary embodiments, the dosage for irinotecan alone is 0.1-100 mg/kg, a non-limiting dosage is 1-10 mg/kg per day. In some exemplary embodiments, the dosage for combination of ethacridine and quinidine is 0.1-50 mg/kg and 0.1-50 mg/kg per day, respectively, a non-limiting dosage is 1-10 mg/kg per day for ethacridine and 0.5-10 mg/kg per day for quinidine. In some exemplary embodiments, the dosage for combination of ethacridine and HEAQ is 0.1-50 mg/kg and 0.1-100 mg/kg per day, respectively, a non-limiting dosage is 1-10 mg/kg per day for ethacridine and 1-50 mg/kg for HEAQ. In some exemplary embodiments, the dosage for combination of ethacridine and propantheline is 0.1-50 mg/kg and 0.1-50 mg/kg per day, respectively, a non-limiting dosage is 1-10 mg/kg per day for ethacridine and 0.1-5 mg/kg for propantheline. In some exemplary embodiments, the dosages of treating PH is the same or different from the dosages for treating cancer. The routine of administration to treat PH may be oral, parenteral, iv, inhalation, intranasal while oral, or parenteral, i.v. or intratumoral administration for treatment of cancer. In some embodiments, PAH treatment is via inhalation or intranasal administration.

The exact dosage will depend upon the route of administration, the form in which the compound is administered, the subject to be treated, the body weight of the subject to be treated, and the preference and experience of the attending physician.

In some embodiments, improvement in a patient's condition, as compared to an untreated control subject, is noted within about a day, a week, two weeks, or within about one month after the first treatment is administered. By way of example, improvements in a patient's condition may include, but is not limited to an improvement in clinical symptoms, test results, histopathological findings, quality of life, tumor reduction or decreased growth and spread, and longevity.

### Advantages and Applications

The disclosed compositions and methods have several advantages and applications that are not taught or suggested in the art. For example, the disclosed methods include treatment of PAH including idiopathic PAH, familial PAH and other forms of hypertension, with a therapeutic composition comprising one or more HIF-2α inhibitors ethacridine, quinidine, and/or analogs of quinidine such as HEAQ or HEAQ analog.

The disclosed methods include treatment of cancer including renal cell carcinoma (ccRCC), glioblastoma multiforme (GBM), and other cancer forms cancer induced by HIF-2α overactivation, or characterized by HIF-2α overexpression. The methods include administering a therapeutic composition comprising one or more HIF-2α inhibitors ethacridine, quinidine, and/or analogs of quinidine such as HEAQ and/or HEAQ analogs, propantheline, etoposide, irinotecan and/or analogs thereof.

Quinidine and its analogs, ethacridine, propantheline, etoposide, irinotecan as well as quinidine or its analog such as HEAQ and ethacridine combination, quinidine or its analog in combination with etoposide or its analog, quinidine or its analog in combination with irinotecan or its analog; ethacridine or its analog in combination with propantheline or its analog, ethacridine or its analog in combination with etoposide or its analog, ethacridine or its analog in combination with irinotecan or its analog; etoposide or its analog in combination with propantheline or its analog, etoposide or its analog in combination with irinotecan or its analog; propantheline or its analog in combination with irinotecan or its analog target the mechanisms underlying pulmonary vascular remodeling via inhibition of HIF-2a-mediated occlusive vascular remodeling. Thus, these drugs inhibit and even reverses pulmonary vascular remodeling and PAH leading to increased survival.

HIF-2α activation is the key driver of RCC and GBM, thus these drugs target the underlying mechanism of RCC and GBM as well as pheochromocytomas and paragangliomas.

### EXAMPLES

The following Examples are illustrative and should not be interpreted to limit the scope of the claimed subject matter.

### Example 1. Methods and Compositions for the Treatment of Pulmonary Hypertension an Cancer

Pulmonary hypertension (PH) including pulmonary artery hypertension (PAH) is characterized by excessive vasoconstriction and obliterative vascular remodeling that leads to right heart failure (RHF) and death (1-3). The histopathological features of PAH include thickening of the medial and adventitial layers of pulmonary vasculature, and the formation of vascular occlusions and plexiform lesions (4-7). Although significant advances have been made in understanding the pathogenesis of PAH in the past decade, no effective therapy has been developed to reverse pulmonary vascular remodeling, inhibit RHF and promote survival of PAH patients. Current therapies mainly target the abnormalities in vasoconstriction in the prostacyclin, nitric oxide and endothelin signaling pathways, but not the obliterative vascular remodeling abnormalities. Hence, only modest improvements are achieved in PAH morbidity and mortality (8-10). Therefore, novel therapeutic agents are urgently needed for PAH patients.

We have recently reported the unprecedented mouse model of PAH [Tie2Cre-mediated disruption of Egln1, encoding hypoxia inducible factor (HIF) prolyl hydroxylase 2 (PHD2), designated Egln1^{Tie2Cre}] with progressive obliterative vascular remodeling including vascular occlusion and plexiform-like lesion and right heart failure, which recapitulates many features of clinical PAH including idiopathic PAH (IAPH) (11). We also show that PHD2 expression was diminished in endothelial cells (ECs) of occlusive pulmonary vessels in IPAH patients (11). Employing the Egln1/Hif2a^{Tie2Cre} double knockout mice, we demonstrated that HIF-2α activation secondary to PHD2 deficiency is responsible for the severe PAH phenotype in Egln1^{Tie2Cre} mice. Furthermore, employing a HIF-2α inhibitor C76, which is believed to inhibit HIF-2α but not HIF-1α translation, we show that pharmacological inhibition of HIF-2α in various animal models of PH inhibits/reverses pulmonary vascular remodeling, inhibits PH and right heart failure (12).

HIFs are key transcription factors mediating adaptive responses to hypoxia and ischemia (13, 14). Given that the cells are frequently in a hypoxic state in the tumor microenvironment, These adaptive mechanisms are hijacked by cancer to help tumors survive and grow and thus targeting HIF signaling has great potential in treating cancer. In 90% of the Clear-cell renal cell carcinoma (ccRCC), the most common form of kidney cancer, the tumor suppressor protein von Hippel-Lindau is not functional and thus HIFs are stabilized (15). Especially, HIF-2α is activated and plays an essential role in ccRCC evident by a) all pVHL-null ccRCC maintain HIF2α expression (16); b) HIF2α function is required for ccRCC xenograft growth (15, 17); c) polymorphisms in EPAS1/HIF2α are associated with increased ccRCC risk in genome-wide association studies (18); and d) expression of HIF-2α in clear-cell renal cell carcinoma independently predicts overall survival in patients (19, 20). Thus, targeting HIF-2α may be an effective therapeutic approach for ccRCC.

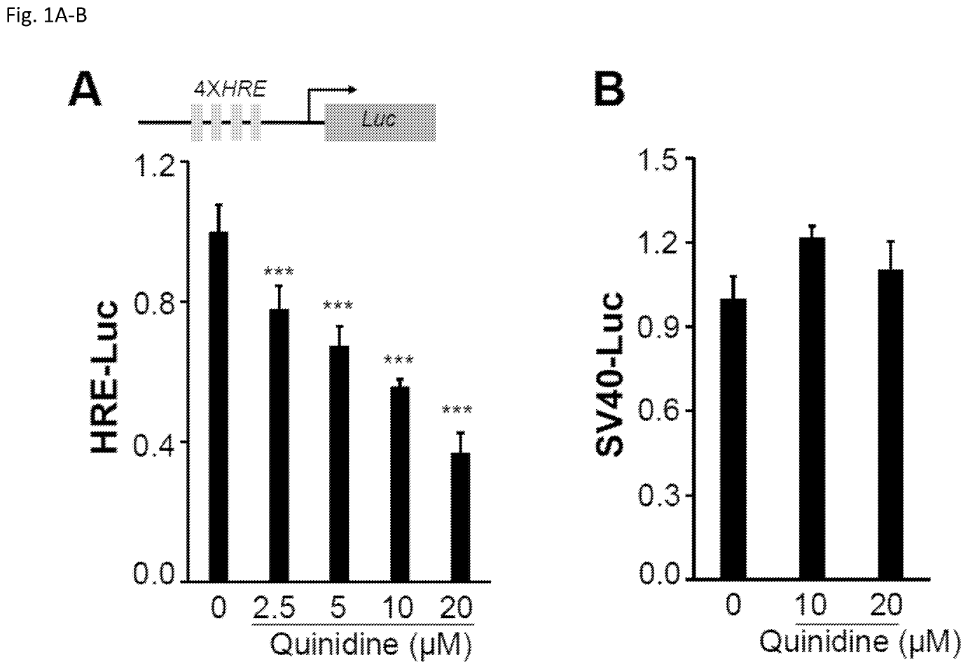

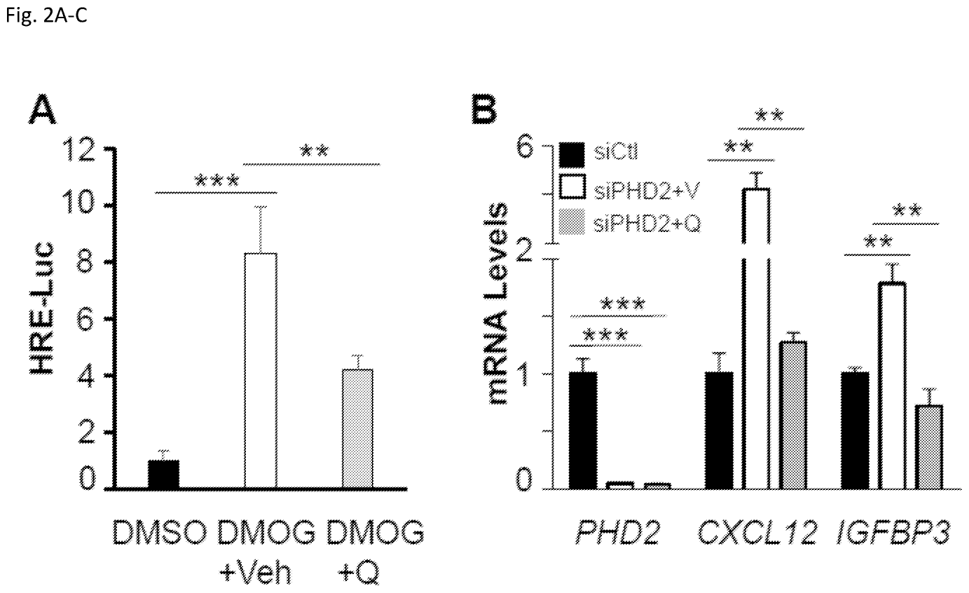

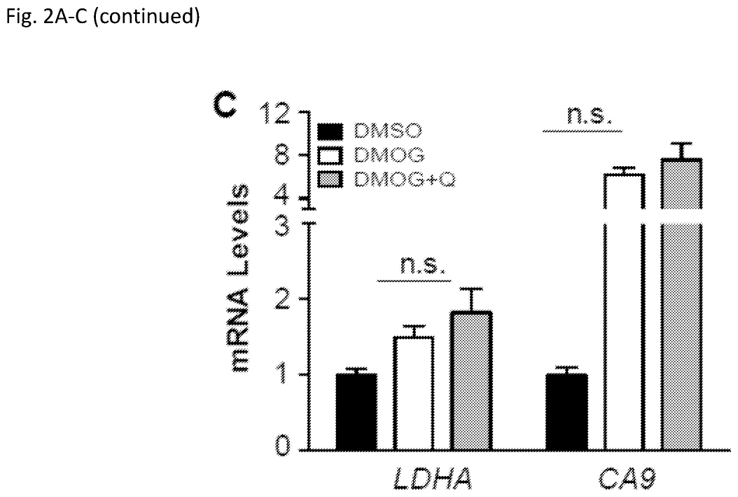

To identify FDA-approved drug(s) that could also inhibit HIF-2α and thereby provide a potential novel therapeutic agent(s) for treatment of PH and cancer (e.g., ccRCC), we carried out high-throughput screening of the Prestwick Chemical Library of FDA-approved drugs (1200 compounds) employing stable hypoxia response element (HRE)/luciferase-expressing human renal cancer cell line 786-O, which are Von Hippel Lindau (VHL) deficient leading to HIF-α (mainly HIF-2α as HIF-1α expression is silenced in this cell line) activation. Quinidine hydrochloride monohydrate (quinidine) was identified and validated as an inhibitor of HIF-2αα evident by inhibition of HRE/luciferase activity in a dose-dependent manner (FIG. 1A) without obvious toxicity in 786-O cells (FIG. 1B). In primary culture of human lung microvascular endothelial cells (HLMVECs), quinidine inhibited HRE/luciferase activity activated by Dimethyloxalylglycine (DMOG) treatment which is a well-known HIF-a stabilizer (FIG. 2A). Quinidine also inhibited expression of HIF-2α target genes in Egln1-deficient HLMVECs (FIG. 2B), but not expression of HIF-1α-specific target genes LDHA and CA9 in human pulmonary arterial smooth muscle cells (hPASMCs) (FIG. 2C). Western blotting show that quinidine didn't inhibit expression of HIF-2α or HIF-1α protein levels (FIG. 2D). Together, these data demonstrate Quinidine is a potent HIF-2α but not HIF-1α inhibitor.

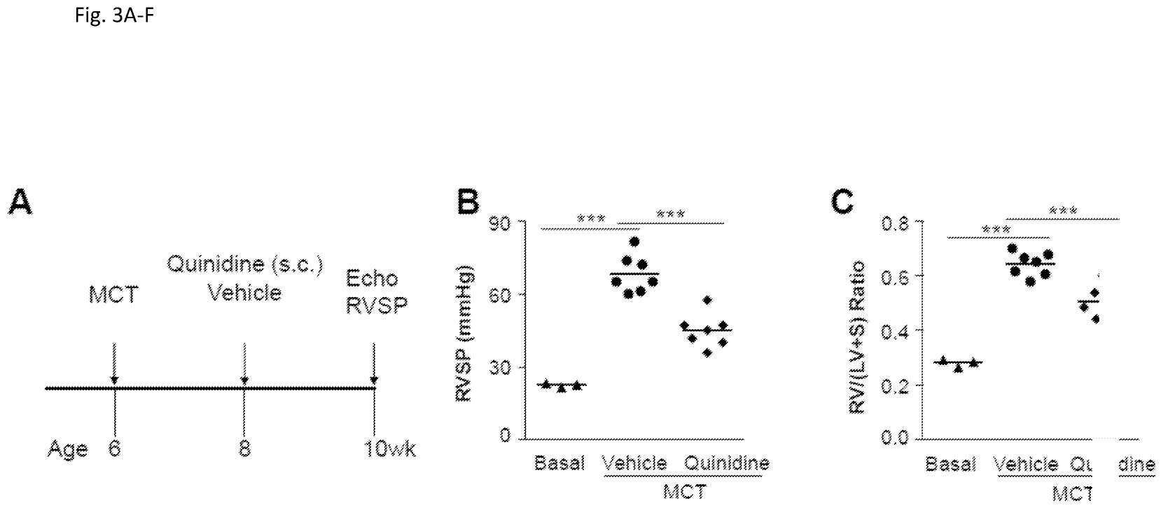

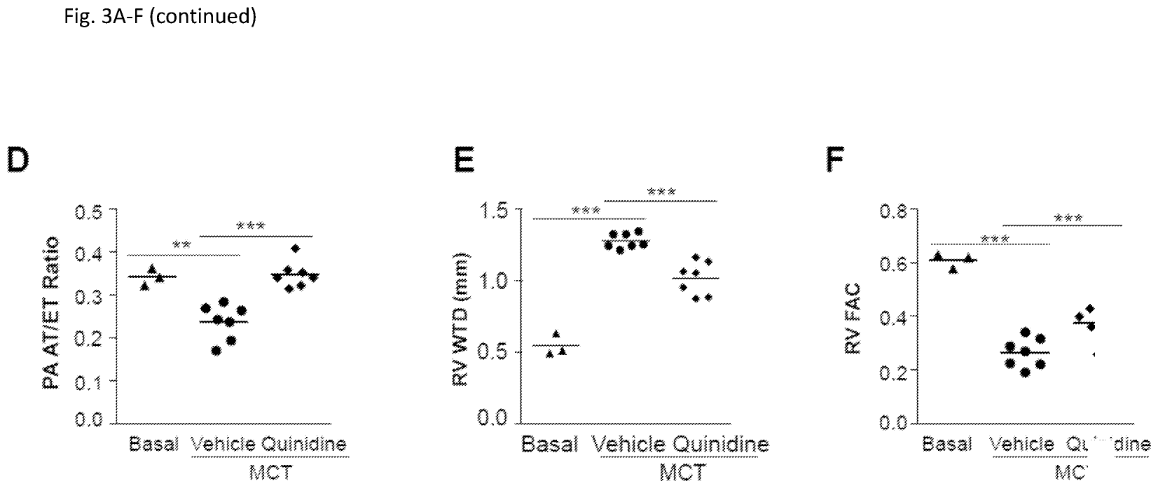

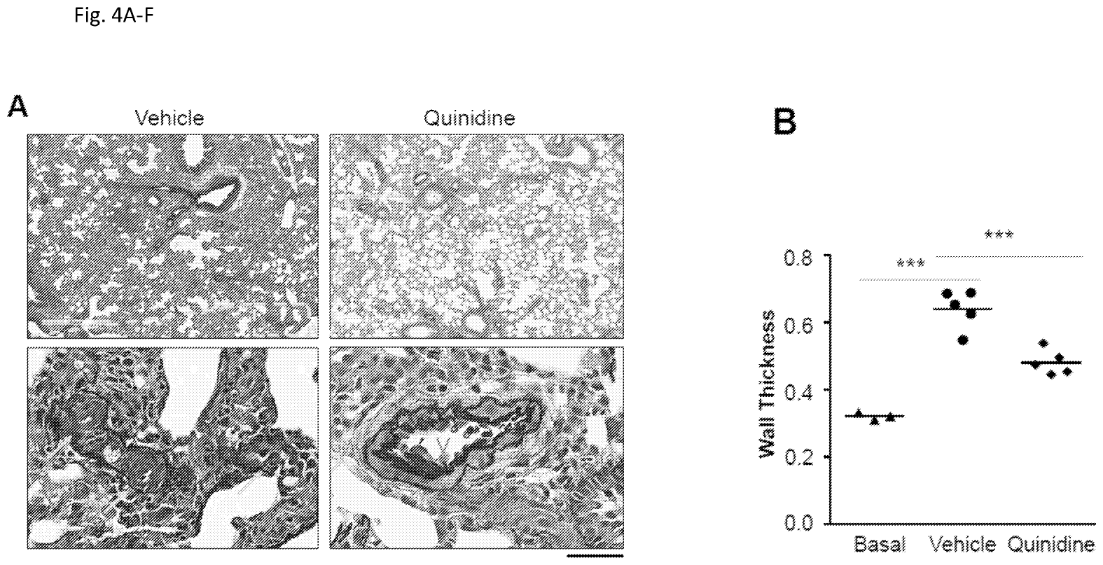

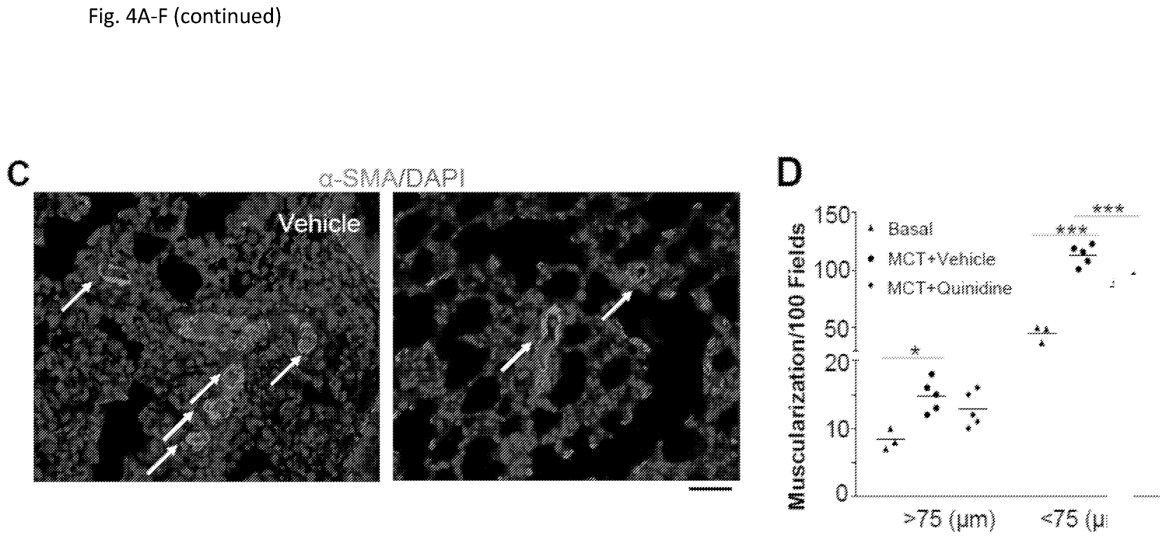

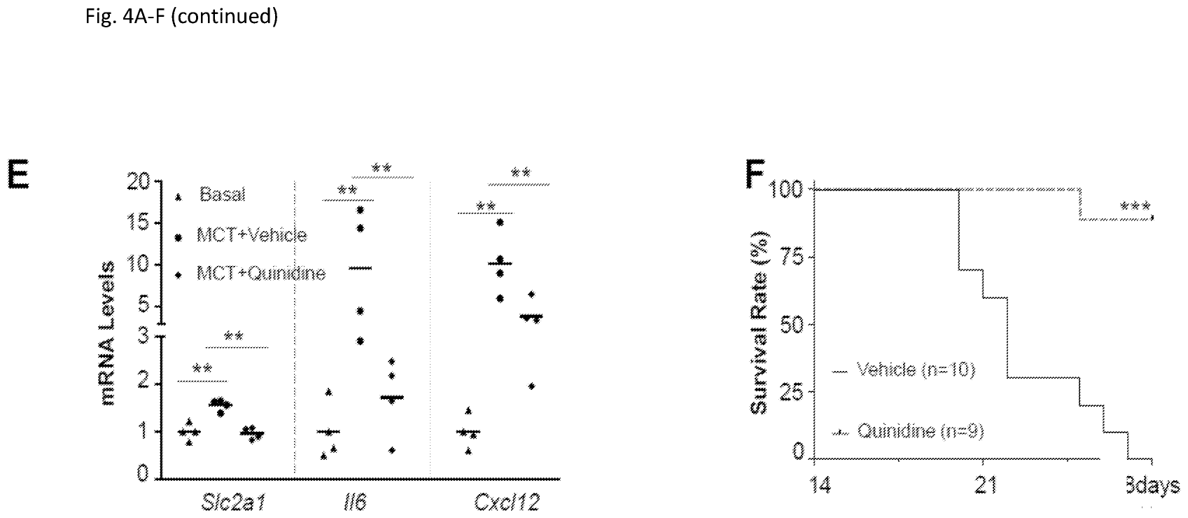

We next examined the efficacy of Quinidine treatment in a rat PH model. At 2-weeks post-monocrotaline challenge, the rats were treated with either Quinidine [(Quinidine gluconate, Eli Lilly and Co., 2.4 mg/rat/day via s.c. osmotic pump (ALZET), i.e. 10-12 mg/kg body weight per day] or vehicle for another 2 weeks (FIG. 3A). Quinidine treatment inhibited PAH evident by marked decrease of right ventricular systolic pressure (RVSP) and RV hypertrophy (FIG. 3B, C), and restored pulmonary artery function (FIG. 3D). Cardiac measurements by echocardiography also showed that Quinidine treatment decreased RV wall thickness (FIG. 3E) and improved RV contractility (FIG. 3F). Pulmonary vascular remodeling including pulmonary artery wall thickening and muscularization of distal pulmonary arteries was also markedly attenuated by Quinidine treatment (FIG. 4A-4D). Expression of HIF-2α downstream target genes was normalized or markedly inhibited in lungs of rats treated with Quinidine compared with vehicle (FIG. 4E). Remarkably, while all rats in the vehicle group had died by day 28 post-MCT challenge with a lethal dose, quinidine treatment resulted in a 90% survival rate at this time (FIG. 4F). Taken together, these data demonstrate that the FDA-approved anti-arrhythmia and anti-malaria drug, Quinidine, could selectively target HIF-2α and reverse aberrant vascular remodeling and right heart failure in the setting of PAH.

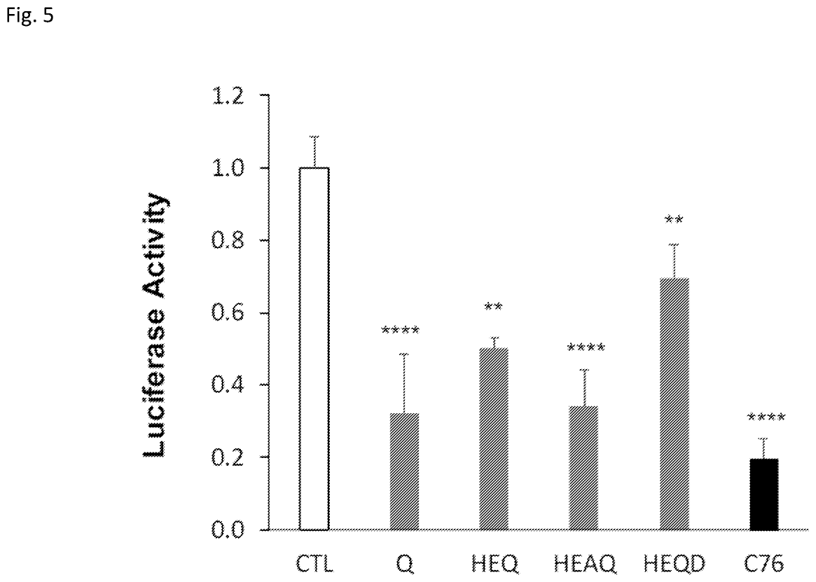

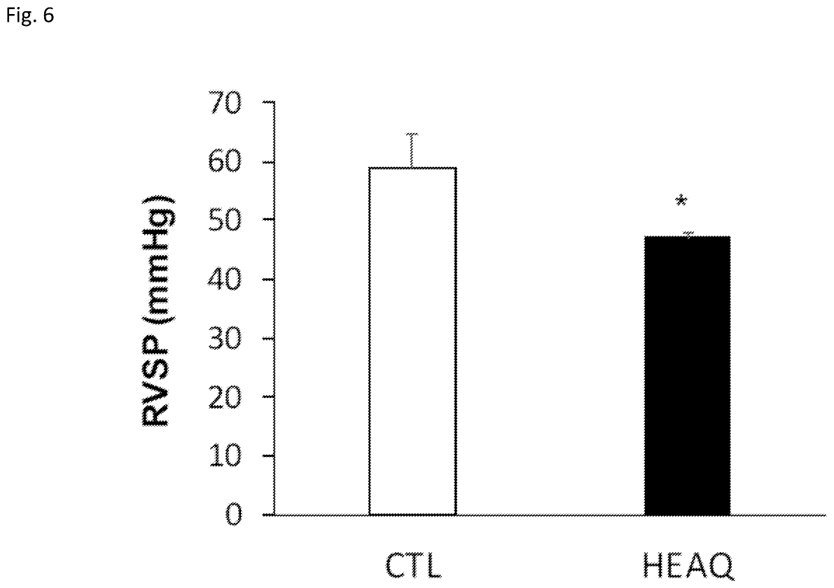

We also determined the inhibitory effects of Quinidine analogs on HIF-2α activity using the stable HRE/luciferase-expressing 786-O cell line. As shown in FIG. 5, hydroxyethylapoquinine (HEAQ) could inhibit HIF activity as efficiently as Quinidine. HEAQ has very low human ether-à-go-go-related gene (hERG) channel inhibition activity (IC50>90 uM) compared to Quinidine (IC50=4 uM) (21), indicating it may have less cardiac arrhythmia effects in patients. In vivo animal study, we observed that HEAQ treatment (oral, 20 mg/kg once a day) attenuate PH in MCT-rats (FIG. 6). HEAQ is a potential drug for PH treatment with better safety profile.

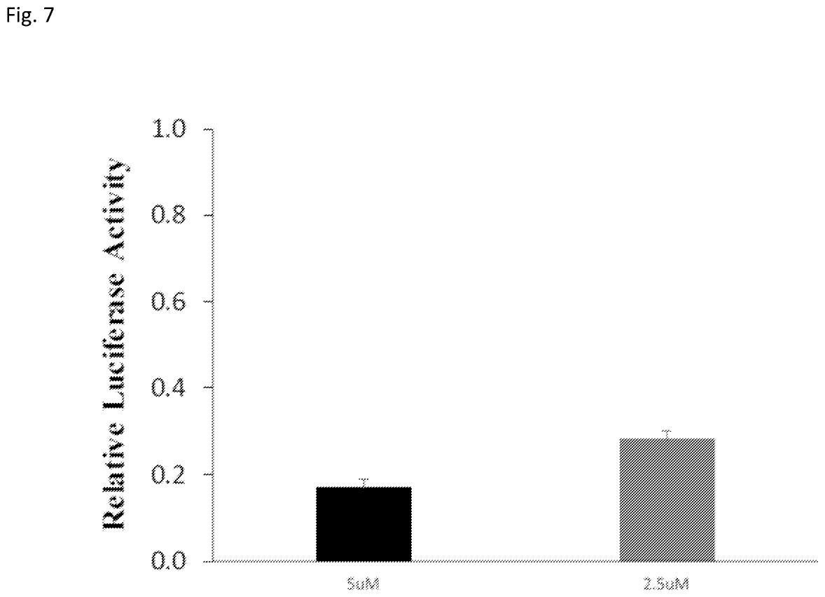

Additionally, our screening has also identified ethacridine, etoposide, methantheline, and irinotecan as potent HIF α inhibitors which were confirmed in cultured 786-O cells (FIG. 7, and Table 1). Ethacridine at 2.5 uM concentration could inhibit 70% of HIF-α activity similar to 20 uM of Quinidine (FIG. 7). etoposide, methantheline, and irinotecan at 1.25 uM, 2.5 uM, 2.5 uM, respectively also inhibited 65%-77% of HIF-α activity without clear toxicity (Table 1). We also determined the effects of these inhibitors on the expression of PH-causing genes in primary cultures of human lung microvascular ECs. As shown in Table 2, ethacridine (20 and 40 uM) treatment markedly inhibited DMOG-induced expression of PDGF-B and endothelin-1 although it induced SDF-1 expression whereas propantheline mainly inhibited SDF-1 expression; etoposide treatment markedly inhibited PDGFB and endothelin-1 expression; irinotecan inhibited PDGFB, endothelin-1 and SDF1 expression.

TABLE 1
Validation of additional HIF inhibitors in 786-O cells

| | HRE-Luc | SV40-Luc |
|---|---|---|
| | (of control) | (of control) |
| Etoposide (1.25 uM) | 0.34 | 0.76 |
| Methantheline (2.5 uM) | 0.31 | 0.86 |
| Irinotecan(2.5 uM) | 0.23 | 0.99 |

As shown in Table 1 above, at the indicated concentration, these compounds markedly inhibit HIF activity (HR-Luc) without significant toxicity with SV40-luciferase activities close to control levels.

TABLE 2
The effects of HIF inhibitors on expression of PH-causing genes in HLMVECs

| PH- | | | | | | |
|---|---|---|---|---|---|---|
| causing | Control | DMOG | Ethacridine | Etoposide | Propantheline | Irinotecan |
| factors | (DMSO) | (DMSO) | (20 & 30 uM) | (20 & 40 uM) | (40 uM) | (20 & 40 uM) |
| PDGF-B | 1.00 | 2.05 ± 0.33 | 0.52 ± 0.31*** | 0.77 ± 0.37** | 1.55 ± 0.44 | 1.16 ± 0.15** |
| Endothelin-1 | 1.00 | 2.36 ± 0.23 | 0.12 ± 0.07**** | 0.47 ± 0.50*** | 1.70 ± 0.45* | 0.73 ± 0.46*** |
| SDF1 | 1.00 | 1.80 ± 0.06 | 12.9 ± 8.1* | 1.25 ± 0.80 | 0.53 ± 0.37** | 0.64 ± 0.10**** |

Regarding Table 2, human lung microvascular endothelial cells (ECs) were treated with 2-Dimethyloxalylglycine (DMOG, PHD inhibitor) and either vehicle (DMSO) or various HIF inhibitors for 24 h. RNA was isolated for quantitative RT-PCR analysis. DMOG treatment alone markedly induced expression of PDGF-B and Endothelin-1 as well as SDF1. Data are expressed as mean±SD. *P<0.05; **P<0.01; ***P<0.001; ****P<0.0001 as compared to DMOG group.

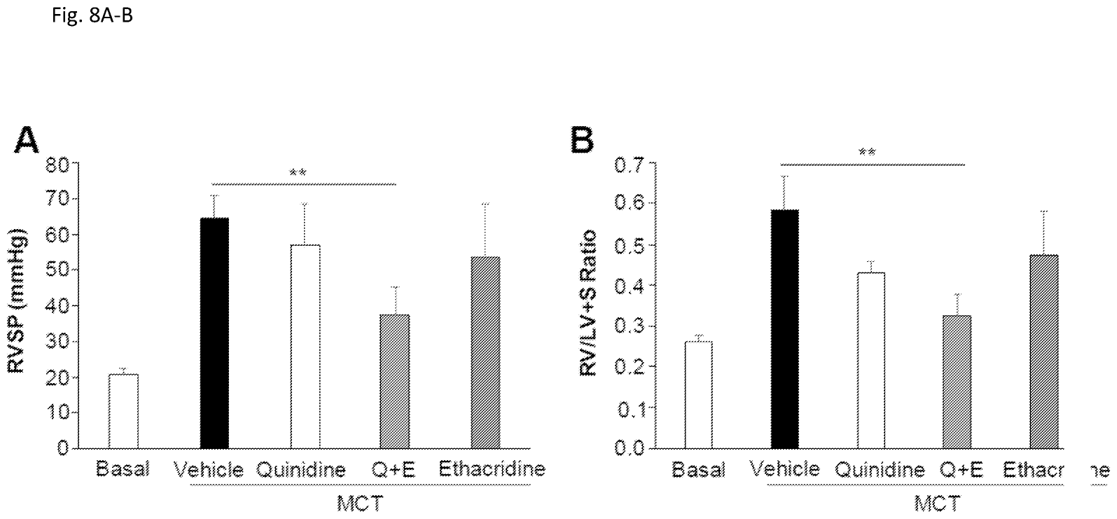

We next tested the combination effects of ethacridine and quinidine to determine whether relative low doses of both drugs could synergistically inhibit HIF-α and PH development leading to enhanced therapeutic effects whereas reduced side effects. As shown in FIG. 8, co-treatment with quinidine (10 mg/kg, oral, twice a day) and ethacridine (7.5 mg/kg, i.p. twice a day) markedly inhibited PH and RV hypertrophy evident by decreased RVSP and RV/LV+septum ratio. But, neither quinidine nor ethacridine at these low doses didn't inhibit PH. Thus, quinidine and its analog HEAQ, and Ethacridine as well as combination of ethacridine and either quinidine or HEAQ or combination of their analogs are potential novel therapeutics for treatment PAH. They are also likely novel agents for cancer treatment such as ccRCC.

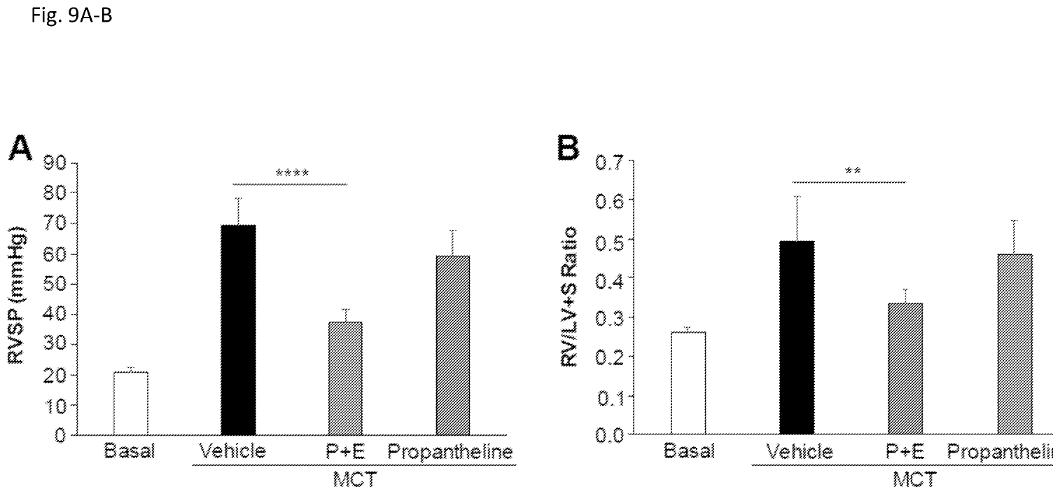

As ethacridine inhibits strongly expression of PDGFB and endothelin-1 but induces SDF1 expression whereas propantheline mainly inhibit SDF1 expression (Table 2), we addressed if ethacridine and propantheline have synergistic effect on inhibition of PH progression. Co-treatment with ethacridine (7.5 mg/kg, i.p., twice a day) and propantheline (1 mg/kg, i.p., twice a day) at 2 weeks post-MCT challenge markedly inhibited pulmonary hypertension indicative of RVSP (FIG. 9A) and RV hypertrophy evident by RV/LV+S ratio (FIG. 9B).

### REFERENCES

1. McLaughlin V V, Archer S L, et al. ACCF/AHA 2009 expert consensus document on pulmonary hypertension: a report of the American College of Cardiology Foundation Task Force on Expert Consensus Documents and the American Heart Association: developed in collaboration with the American College of Chest Physicians, American Thoracic Society, Inc., and the Pulmonary Hypertension Association. Circulation. 2009; 119: 2250-2294.
2. Voelkel N F, Cool C. Pathology of pulmonary hypertension. Cardiol Clin. 2004; 22: 343-51, v.
3. Pietra G G, Capron F, Stewart S, Leone O, Humbert M, Robbins I M, Reid L M, Tuder R M. Pathologic assessment of vasculopathies in pulmonary hypertension. J Am Coll Cardiol. 2004; 43: 25S-32S.
4. Meyrick B. The pathology of pulmonary artery hypertension. Clin Chest Med. 2001; 22: 393-404, vii.
5. Stacher E, Graham B B, Hunt J M, Gandjeva A, Groshong S D, McLaughlin V V, Jessup M, Grizzle W E, Aldred M A, Cool C D, Tuder R M. Modern age pathology of pulmonary arterial hypertension. Am J Respir Crit Care Med. 2012; 186: 261-272.
6. Farber H W. The status of pulmonary arterial hypertension in 2008. Circulation. 2008; 117: 2966-2968.
7. Sutendra G, Michelakis E D. Pulmonary arterial hypertension: challenges in translational research and a vision for change. Sci Transl Med. 2013; 5: 208sr5.
8. McLaughlin V V, Shah S J, Souza R, Humbert M. Management of pulmonary arterial hypertension. J Am Coll Cardiol. 2015; 65: 1976-1997.
9. Ryan J J, Marsboom G, Archer S L. Rodent models of group 1 pulmonary hypertension. Handb Exp Pharmacol. 2013; 218: 105-49.
10. Gomez-Arroyo J, Saleem S J, Mizuno S, Syed A A, Bogaard H J, Abbate A, Taraseviciene-Stewart L, Sung Y, Kraskauskas D, Farkas D, Conrad D H, Nicolls M R, Voelkel N F. A brief overview of mouse models of pulmonary arterial hypertension: problems and prospects. Am J Physiol Lung Cell Mol Physiol. 2012; 302: L977-91.
11. Dai Z, Li M, Wharton J, Zhu M M, Zhao Y-Y. Prolyl-4 Hydroxylase 2 (PHD2) Deficiency in Endothelial Cells and Hematopoietic Cells Induces Obliterative Vascular Remodeling and Severe Pulmonary Arterial Hypertension in Mice and Humans Through Hypoxia-Inducible Factor-2α. Circulation 2016; 133:2447-58.
12. Dai Z, Zhu M M, Peng Y, Machireddy N, Evans C E, Machado R, Zhang X, Zhao Y-Y. Therapeutic Targeting of Vascular Remodeling and Right Heart Failure in PAH with HIF-2α Inhibitor. Am J Respir Crit Care Med 2018; rccm.201710-2079OC.doi:10.1164/rccm.201710-2079OC.
13. Majmundar A J, Wong W J, Simon M C. Hypoxia-inducible factors and the response to hypoxic stress. Mol Cell. 2010; 40: 294-309.
14. Semenza G L. Hypoxia-inducible factors in physiology and medicine. Cell. 2012; 148: 399-408.
15. Shen C, Kaelin W G, Jr. The VHL/HIF axis in clear cell renal carcinoma. Semin Cancer Biol 2013; 23:18-25.
16. Gordan J D, Lal P, Dondeti V R, Letrero R, Parekh K N, Oquendo C E, et al. HIF-alpha effects on c-Myc distinguish two subtypes of sporadic VHL-deficient clear cell renal carcinoma. Cancer Cell 2008; 14:435-46.
17. Keith B, Johnson R S, Simon M C. HIF1alpha and HIF2alpha: sibling rivalry in hypoxic tumour growth and progression. Nat Rev Cancer 2012; 12:9-22.
18. Purdue M P, Johansson M, Zelenika D, Toro J R, Scelo G, Moore L E, et al. Genome-wide association study of renal cell carcinoma identifies two susceptibility loci on 2p21 and 11q13.3. Nat Genet 2011; 43:60-5.
19. Schodel J, Grampp S, Maher E R, Moch H, Ratcliffe P J, Russo P, Mole D R. 2016. Hypoxia, hypoxia-inducible transcription factors, and renal cancer. Eur Urol 69: 646-657.
20. Gkagkalidis K, Kampantais S, Dimitriadis G, Gourvas V, Kapoukranidou D, Mironidou-Tzouveleki M. Expression of HIF-2α in clear-cell reneal cell carcinoma independently predicts overall survival. Med Mol Morphol. 2020 Mar 26. doi: 10.1007/s00795-020-00249-3.
21. Sanders N G, Meyers D J, Sullivan D J. Antimalarial efficacy of hydroxyethylapoquinine (SN-119) and its derivatives. Antimicrob Agents Chemother. 2014; 58(2):820-7.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method of treating pulmonary hypertension in a subject in need thereof, the method comprising:
(a) administering to the subject an effective amount of one of (1)-(5):
(1) quinidine, and/or an analog thereof, such as hydroxyethylapoquinine (HEAQ);
(2) ethacridine and/or an analog thereof;
(3) etoposide and/or analog thereof;
(4) propantheline and/or analog thereof; or
(5) irinotecan and/or analog thereof;
(b) or administering to the subject an effective amount of one of (1)-(17):
(1) quinidine or an analog thereof such as HEAQ or a HEAQ analog thereof, and administering to the subject an effective amount of ethacridine or analog thereof, before, concurrently with, or after administering to the subject an effective amount of quinidine or an analog thereof such as HEAQ or HEAQ analog thereof;
(2) quinidine or an analog thereof such as HEAQ or a HEAQ analog thereof, and administering to the subject an effective amount of propantheline or analog thereof, before, concurrently with, or after administering to the subject an effective amount of quinidine or an analog thereof such as HEAQ or HEAQ analog thereof;
(3) quinidine or an analog thereof such as HEAQ or analog thereof, and administering to the subject an effective amount of etoposide or analog thereof, before, concurrently with, or after administering to the subject an effective amount of quinidine or an analog thereof such as HEAQ or HEAQ analog thereof;
(4) quinidine or an analog thereof such as HEAQ or analog thereof, and administering to the subject an effective amount of irinotecan or analog thereof, before, concurrently with, or after administering to the subject an effective amount of quinidine or an analog thereof such as HEAQ or HEAQ analog thereof;
(5) ethacridine or an analog thereof, and administering to the subject an effective amount of propantheline or analog thereof, before, concurrently with, or after administering to the subject an effective amount of ethacridine or an analog thereof;
(6) ethacridine or an analog thereof, and administering to the subject an effective amount of etoposide or analog thereof, before, concurrently with, or after administering to the subject an effective amount of ethacridine or an analog thereof;
(7) ethacridine or an analog thereof, and administering to the subject an effective amount of irinotecan or analog thereof, before, concurrently with, or after administering to the subject an effective amount of ethacridine or an analog thereof;
(8) propantheline or an analog thereof, and administering to the subject an effective amount of etoposide or analog thereof, before, concurrently with, or after administering to the subject an effective amount of propantheline or an analog thereof;
(9) propantheline or an analog thereof, and administering to the subject an effective amount of irinotecan or analog thereof, before, concurrently with, or after administering to the subject an effective amount of propantheline or an analog thereof;
(10) etoposide or an analog thereof, and administering to the subject an effective amount of irinotecan or analog thereof, before, concurrently with, or after administering to the subject an effective amount of etoposide or an analog thereof;
(11) etoposide or an analog thereof, and administering to the subject an effective amount of irinotecan or analog thereof, before, concurrently with, or after administering to the subject an effective amount of etoposide or an analog thereof;
(12) quinidine or an analog thereof such as HEAQ, and administering to the subject an effective amount of ethacridine or analog thereof, and administering to the subject an effective amount of propantheline or analog thereof before, concurrently with, or after administering to the subject an effective amount of quinidine or an analog thereof such as HEAQ or HEAQ analog;
(13) quinidine or an analog thereof such as HEAQ, and administering to the subject an effective amount of ethacridine or analog thereof, and administering to the subject an effective amount of etoposide or analog thereof before, concurrently with, or after administering to the subject an effective amount of quinidine or an analog thereof such as HEAQ or HEAQ analog;
(14) quinidine or an analog thereof such as HEAQ, and administering to the subject an effective amount of ethacridine or analog thereof, and administering to the subject an effective amount of irinotecan or analog thereof before, concurrently with, or after administering to the subject an effective amount of quinidine or an analog thereof such as HEAQ or HEAQ analog;
(15) ethacridine or an analog thereof, and administering to the subject an effective amount of propantheline or analog thereof, and administering to the subject an effective amount of etoposide or analog thereof before, concurrently with, or after administering to the subject an effective amount of ethacridine;
(16) ethacridine or an analog thereof, and administering to the subject an effective amount of propantheline or analog thereof, and administering to the subject an effective amount of irinotecan or analog thereof before, concurrently with, or after administering to the subject an effective amount of ethacridine; or
(17) propantheline or an analog thereof, and administering to the subject an effective amount of etoposide or analog thereof, and administering to the subject an effective amount of irinotecan or analog thereof before, concurrently with, or after administering to the subject an effective amount of propantheline.

2. The method of claim 1, wherein the pulmonary hypertension comprises pulmonary artery hypertension (PAH).

3. The method of claim 1, wherein the pulmonary hypertension comprises idiopathic PAH (IPAH).

4. The method of claim 1, comprising administering one or more additional active agent to the subject.

5. The method of claim 4, wherein the additional active agent comprises one or more of a vasodilator such as ambrisentan (Letairis), bocentan (Tracleer), macitentan (Opsumit), riociguat (Adempas), selexipag (Uptravi), sildenafil (Revatio), tadalafil (Adcirca), treprostinil (Orenitram), iloprost tromethamine (Ventavis), treprostinil (Tyvaso), epopostenol sodium (Flolan, Veletri), treprostinil, or a calcium channel blocker.

6. A method of treating cancer in a subject in need thereof, the method comprising: administering to the subject an effective amount of one or more of quinidine and/or an analog thereof, such as hydroxyethylapoquinine (HEAR) and/or analog thereof, ethacridine and/or analog thereof, propantheline and/or analog thereof, etoposide and/or analog thereof, irinotecan and/or analog thereof.

7. The method of claim 6, wherein the cancer is characterized by an increased expression or activity of HIF-2α.

8. The method of claim 6, wherein the cancer comprises one or more of clear cell renal cell carcinoma, glioblastoma multiforma (GBM), pheochromocytoma, and paraganglioma.

9. The method of claim 6 comprising administering one or more additional therapeutic agent to the subject.

10. A pharmaceutical composition comprising:
(a) quinidine or HEAQ or analog thereof and either ethacridine, propantheline, etoposide or irinotecan or analog thereof;
(b) ethacridine or analog thereof and either propantheline, etoposide, or irinotecan or analog thereof;
(c) propantheline or analog thereof and either etoposide, or irinotecan or analog thereof;
(d) etoposide or analog thereof, and irinotecan or analog thereof;
(e) quinidine or HEAQ or analog thereof, and ethacridine or analog thereof, and either propantheline, etoposide or irinotecan or analog thereof;
(f) ethacridine or analog thereof, and propantheline or analog thereof, and either etoposide or irinotecan or analog thereof;
(g) propantheline or analog thereof, and etoposide or analog thereof, and irinotecan or analog thereof; or
(h) one or more of ethacridine, quinidine, HEAQ, propantheline, etoposide, irinotecan, or analog thereof.

11-17. (canceled)

18. The pharmaceutical composition of claim 10, formulated for oral administration.

19. The pharmaceutical composition of claim 10, formulated for parenteral administration.

20. The pharmaceutical composition of claim 10, formulated for inhalation or intranasal administration.
